(19) United States
(12) Patent Application Publication
(10) Pub. No.: US 2006/0276704 A1
(43) Pub. Date: Dec. 7, 2006

(54) Neurophysiological electrode placement apparel

(76) Inventors: William J. McGinnis, Cincinnati, OH (US); Scott A. Metrick, Glenview, IL (US); Jonathan S. Citow, Glencoe, IL (US)

Correspondence Address: LADAS & PARRY LLP, 224 SOUTH MICHIGAN AVENUE, SUITE 1600, CHICAGO, IL, 60604, US

(21) Appl. No.: 11/262,085

(22) Filed: Oct. 29, 2005

Related U.S. Application Data
(63) Continuation-in-part of application No. 11/144,214, filed on Jun. 3, 2005.

(51) Int. Cl. A61B 5/04

(52) U.S. Cl. 600/388

(57) ABSTRACT

A stocking or sleeve for wearing or placing on a part of a subject's body for guiding placement of electrodes during monitoring and evaluating of electroneurophysiological data such as mixed and dermatomal nerve conduction latencies and amplitudes, and spontaneous electromyogram, in both clinical and intraoperative settings. The stocking or sleeve has designed into the fabric positioned apertures corresponding to a specific electrode montage, wherein the electrodes are placed on the surface or just beneath the surface of the skin of a subject at the positions of the apertures. The apertures may be color-coded or otherwise marked, or may be markable, to distinguish particular electrodes for positioning at specific sites on the subject's body. The stocking or sleeve is suitable for all kinds of electrodes. A preferred embodiment of the present invention is the use of the stocking or sleeve with a wireless biosensor electrode. Another preferred embodiment of the present invention is the use of the stocking or sleeve as a component of a system for real-time monitoring of changes in electroneurophysiological data.

### BACKGROUND OF INVENTION

This is a continuation-in-part of pending U.S. Ser. No. 11/244,214, filed on Jun. 3, 2005, and entitled Method Of Using Dermatomal Somatosensory Evoked Potentials In Real-Time For Surgical And Clinical Management which is hereby incorporated by reference.

### FIELD OF THE INVENTION

This invention relates generally to the field of neurophysiology, and particularly to devices for monitoring and evaluating of electroneurophysiological data, particularly for stimulating a subject and recording far field bio-potentials in a subject in both clinical and intraoperative settings.

Elicitation and recording of electrophysiological potentials via electrodes on predetermined sites on the body, such as electrocardiograms (ECG), electromyograms (EMG), and evoked potentials such as somatosensory evoked potentials (SSEP) and dermatomal somatosensory evoked potentials (DSSEP), are all well documented in the medical literature. Somatosensory evoked potentials are neurophysiologic representations of somatosensory pathway function. They are assessed neurophysiologically for latency and amplitude measurements that reflect mixed nerve (both sensory and motor fiber) function. These responses are averaged and a mean mathematical representation is presented as an “evoked response” or “evoked potential.” Generally, mixed nerve SSEPs are robust and easily obtained from peripheral stimulation sites, and their use is well established clinically for evaluating the electrophysiological presentation in patients with neurological symptoms. Anatomically innervated by multiple overlapping nerve roots, SSEPs assess mixed nerve function and cannot be used specifically to identify problems found with individual nerve roots. DSSEPs however are able to assess individual nerve root function.

When a patient undergoes a test of the functional presentation of their nervous system, it is common practice to assess the nerve function by recording of the nerve with an electrical current and record the current transported by the nerve to the spinal cord. The current transported by the nerve to the spinal cord ultimately reaches the location in the brain where cortical control of the nerve is located. If recording electrodes are placed over the spinal cord or over the area of the brain where cortical control of the nerve is located, biopotential amplifiers will record a signal when the signal reaches the electrode. Common practice is to take an averaged sample of the time taken for the signal to reach the electrode and mark the time as the latency, or the time taken for the stimulus to reach the recording electrode. Equipment for obtaining such electrophysiological measurements generally requires manual marking of the latency and requires the user to correlate the measurement and assess the neurological correlation of the finding, a process that can be time-consuming and technically demanding.

A software for evaluating collected electroneurophysiological data, validate quality collection, confirm stimulus-recording placement, compare collected samples to normal based on neurological correlation and provide a comprehensive neurophysiological assessment based on the collected electrophysiological data would be a significant advance over current practice.

It would be highly advantageous to clinicians and surgeons alike to be able to compare and correlate electrophysiological data in real-time while they are being recorded, particularly intraoperatively.

Although obtaining DSSEPs is non-invasive, and relatively inexpensive, the technique is technically demanding, and reproducible results are difficult to obtain. The literature identifies the primary recording site for a dermatomal response as being over the somatosensory cortex. However, signals from the cortex are known to be ambiguous at best in both awake and in anaesthetized patients. Owen et al, (Spine vol. 18, No. 6, pgs 748-754 (1993)) in studying the differences in the levels of the DSSEP and nerve root involvement, report variable results in the peripheral innervations patterns of the dorsal nerve roots in the cervical and lumbar spine. U.S. Pat. No. 5,338,587 addressed the lack of reproducibility of responses detected at the cerebral cortex through static comparisons of transport times (latency) of signals from different stimulating electrodes.

It has been surprisingly found that superior and robust DSSEP waveforms may be obtained at a subcortical recording site. Reproducible high-confidence DSSEP data would be a considerable advance.

Numerous problems are associated with conventional methods of electrode placement. The vast preponderance of recording requires multi-site stimulation and recording montages resulting in multiple electrodes being applied to a single subject, often providing an opportunity for confusion, non-sequential solicitation and protocol breech of electrophysiological data. In a clinical setting, the clinician has visual appreciation of electrode placement and site confirmation, however, with as many as eight paired electrodes, sixteen total electrodes on a single side, logistical coordination can present as a challenge. Further, in the operative suite where multiple agenda's are being implemented and as many as sixty to seventy electrodes are applied, logistical coordination can be a major issue.

Accordingly, there is a need for providing a system that addresses logistical and wire coordination issues. Additionally, there is a need for providing a means of assuring correct anatomical and physiological electrode stimulation and recording placement site in the upper and lower extremities.

The prior art does not teach a simple, easily manufactured, stocking, sleeve or apparel having positioned apertures corresponding to a specific electrode montage to help guide placement of electrodes at sites on the subject's limb for stimulating muscles or nerves, and recording electroneurophysiological data from the subject in clinical and intraoperative settings.

Such a device would find immediate use for in neurophysiological procedures, and particularly in real-time recording and monitoring of changes in electroneurophysiological data.

### SUMMARY OF THE INVENTION

This invention is directed to a manufactured stocking or sleeve or item of apparel for wearing or wrapping around a part of a subject's body during a neurological procedure, having positioned apertures corresponding to a specific electrode montage for guiding placement of electrodes, wherein the electrodes are placed on the surface or just beneath the surface of the skin of a subject at the positions of the apertures.

Accordingly, in one aspect, this invention provides a stocking or sleeve worn on or is wrapped around a leg, an arm, a hand, an upper part of a subject's trunk or a lower part of the trunk having apertures corresponding with an electrode montage for guiding placement of electrodes. In a preferred embodiment the apertures are marked or distinguished to aid placement of electrodes. In a preferred embodiment, the apertures are color-coded.

In another aspect, this invention provides a system for monitoring and evaluating a neurophysiological response in a mammalian subject, comprising means for collecting, analyzing, correlating and reporting electro-neurophysiological data in real-time, wherein the stocking or sleeve having apertures corresponding with an electrode montage for guiding placement of electrodes is a component in the system.

In yet another aspect, this invention provides a system a system for comparing and evaluating elicited bio-potentials in real-time, for example electrocardiogram data, electromyogram data or evoked potentials, by a stimulating electrode at a stimulation site on a mammalian subject, the system comprising hardware means for eliciting a signal from a first stimulation site on a subject, receiving and amplifying the signal, and recording a waveform signal, hardware means for automatically digitally converting the waveform signal and software means for assigning numeric values for the absolute amplitude and absolute latency of the waveform signal, hardware and software means for obtaining series of replicated digitally assigned waveform data for the first stimulation site, software means for mathematically conditioning the series of replicated digitally assigned waveform data, obtaining a validated mean value for the waveform data for the first stimulation site, then comparing the validated mean value with protocol-specific and subject-specific normal waveform data, assessing the comparison and noting the deviations of the waveform data from normal data, and software means for serially comparing and evaluating in real-time the changes in the waveform data and saving the comparisons and changes as a function of time. A preferred embodiment further comprises means for carrying out the foregoing with respect to two or more different stimulations sites on the subject, and for comparing and evaluating the changes in the waveform data and saving the serially obtained comparisons and evaluations as a function of time, and moreover, comprising the means for recording the stimulation signal at a subcortical recording site on the subject, and furthermore, comprising the means for correlating more than one set of bio-potentials from the same subject. In a highly preferred embodiment, the above further comprises the inventive sleeve or stocking having positioned apertures corresponding to a specific electrode montage for electrode placement.

In yet another aspect of the invention is provided a bio-potential signal acquisition system comprising the hardware and software means of the foregoing and moreover further comprising the aforementioned stocking or sleeve.

In a further aspect, this invention provides a method of comparing and evaluating in real-time bio-potentials elicited by a stimulating electrode at a stimulation site on a mammalian subject, and moreover, further comprising placing electrodes on the subject by means of a stocking or sleeve having positioned apertures corresponding to a specific electrode montage for guiding placement of electrodes, wherein electrodes are placed on the surface or just beneath the surface of the skin of a subject at the positions of the apertures.

In another aspect, this invention provides chart for use with the stocking or sleeve of the aforementioned.

### BRIEF DESCRIPTION OF THE DRAWINGS

Having thus described the invention in general terms, reference will now be made to the accompanying drawings, which are not necessarily drawn to scale, and wherein:

FIG. 1 is a pictorial view of the distribution of dermatomes showing the position of spinal roots L1 (1), L2 (2), L3 (3), L4 (4), L5 (5), and S1 (6).

FIGS. 2A and 2B represent, respectively, a posterior view and a lower limb view of a stocking showing the positions of the apertures for placement of pairs of snap-on electrodes in a particular montage in which electrodes are placed over the peroneal nerve, at the spinal root L4 and over the posterior tibial nerve, in accordance with one of the preferred embodiments of the invention.

FIG. 3 represents an anterior view of a stocking showing the positions of apertures for placement of snap-on electrodes at spinal roots L2, L3 and L5.

FIGS. 4A, 4B and 4C show, respectively, an anterior view, a posterior view and an inner lower limb of a stocking showing the positions of apertures for placement of pairs of needle electrodes over the quadriceps muscle, the lateral femoral cutaneous nerve, the vastus lateralis nerve, the tibialis anterior muscle, the extensor hallucis longus muscle (EHL), the peroneal nerve, the gastrocnemius muscle and the posterior tibial nerve.

FIGS. 5A, 5B and 5C show, respectively, a posterior view, an inner lower limb view, and an anterior view of a stocking showing the positions of apertures for placement of pairs of snap-on and needle electrodes over the gastrocnemius muscle, the spinal roots S1, L4, L3, L5, the posterior tibial nerve, the quadriceps muscle and the tibialis anterior muscle.

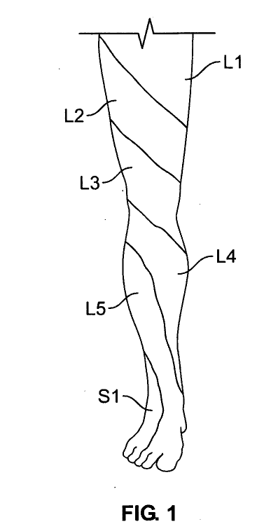

### DETAILED DESCRIPTION

The present invention relates to systems and methods for real-time neurophysiological monitoring as described in U.S. patent application Ser. No. 11/244,214 filed Jun. 3, 2005, the entire contents of which application being incorporated herein by reference.

The present invention provides apparel and especially a stocking or a sleeve for wearing or placing on a subject's body, especially on a subject's limbs, during neurophysiological procedures and neurophysiological monitoring for guidance of placement of electrodes relating to a particular electrode montage, and more particularly, the stocking or sleeve having positioned apertures corresponding to a specific electrode montage, wherein electrodes are placed on the surface or just beneath the surface of the skin of a subject at the positions of the apertures. It is contemplated in one highly preferred embodiment that the inventive stocking or sleeve be designed and manufactured to comprise apertures positioned in the fabric for a specific electrode montage to indicate, guide, and help electrode placement on the subject. It being understood that various different electrode montages may be appropriate in particular circumstances, it is contemplated that various designs of stocking or sleeve or glove or trunk apparel are possible having different distributions of apertures, according to the montage with which the apparel is to be used.

The inventive stocking or sleeve being may be made of any suitable fabric for holding in place. The inventive stocking or sleeve may be suitable for placing on or against a subject's leg, arm, hand, upper torso, lower torso and the like.

One embodiment of the inventive stocking or sleeve comprises color-coded apertures, the color-coding relating to the placement of specific electrodes at specific sites on a subject's body. The inventive stocking or sleeve is suitable for all kinds of electrodes, and for use in both static and real-time neurophysiological recording and monitoring. Related application, U.S. patent application Ser. No. 11/144,214 filed Jun. 3, 2005, which is incorporated herein by reference, describes systems, apparatus and methods for both static and real-time neurophysiological procedures. It is contemplated by the inventors that the inventive stocking or sleeve is a useful component of those systems, apparatus and methods.

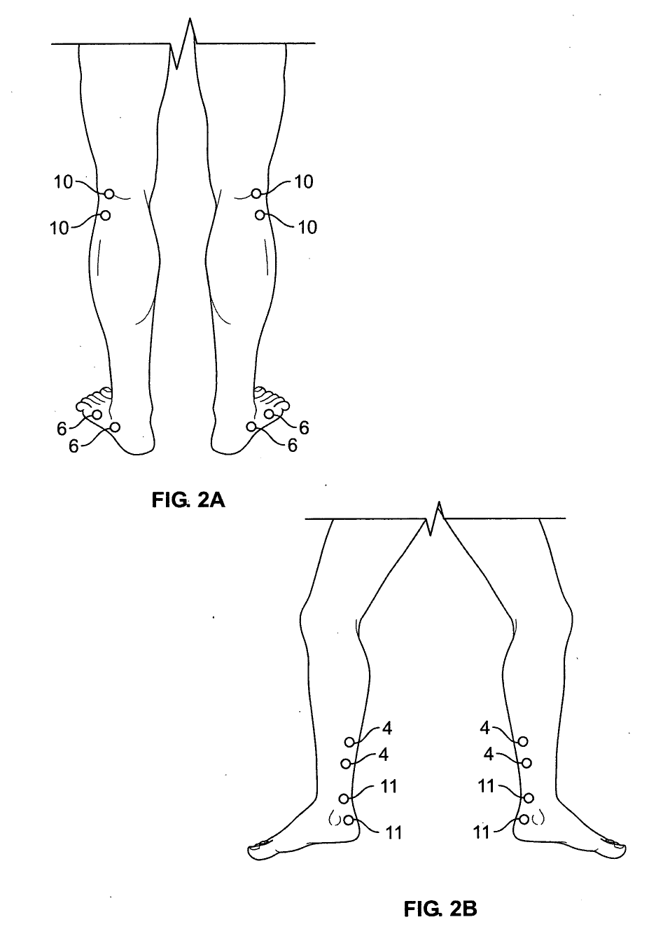

The above described FIGS. 2-5 illustrate the inventive stocking in several embodiments as designed for various electrode montages. In the instant figures, two different sizes of apertures are exemplified to demonstrate use of the inventive stocking when employing snap-on and needle electrodes. Typically, snap-on electrodes are for detecting nerve and spinal root signals, and needle electrodes are used for muscle recording or stimulation. Procedures employing a combination of both snap-on and needle electrodes accommodate procedures in which both muscle and nerve, and spinal root signal recordings are carried out during a single procedure.

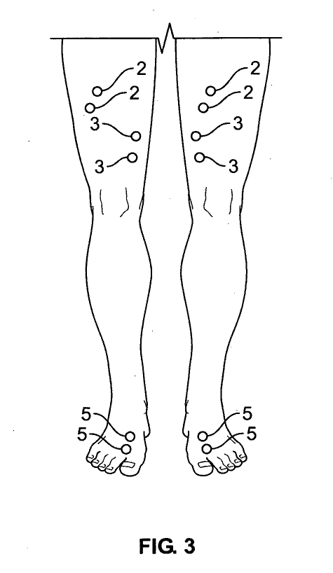

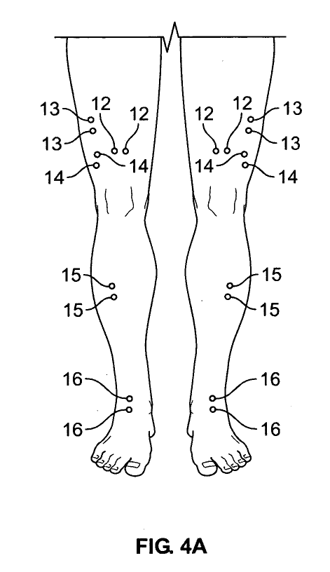

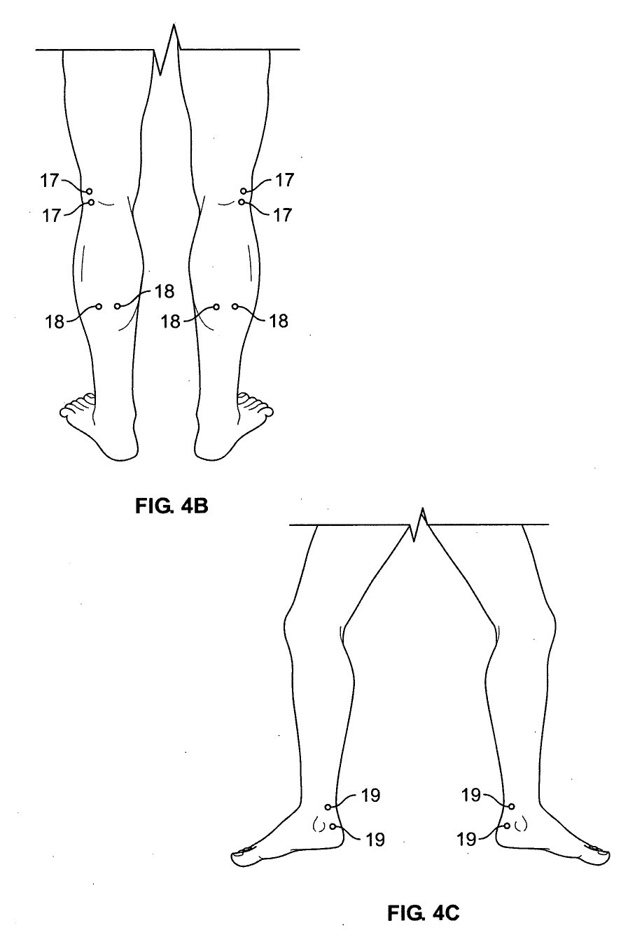

Preferred embodiments of the inventive stocking are shown in FIGS. 2-5. FIGS. 2A, 2B and FIG. 3 illustrate the inventive stocking having large apertures suited for a montage using all snap-on electrodes. FIGS. 2A and 2B represent, respectively, a posterior view and an inner lower limb view, showing the positions of the apertures over the peroneal nerve, (10), at the spinal root L4, (6), and over the posterior tibial nerve (11), in accordance with one of the preferred embodiments of the invention. FIG. 3 represents an anterior view, showing the positions of apertures at spinal roots L2 (2), L3 (3) and L5 (5). FIGS. 4A, 4B and 4C, illustrate the inventive stocking having smaller apertures suited for a montage using all needle electrodes. FIGS. 4A, 4B and 4C show, respectively, an anterior view, a posterior view and an inner lower limb view of a stocking showing the positions of apertures for placement of pairs of needle electrodes over the quadriceps muscle (12), the lateral femoral cutaneous nerve (13), the vastus lateralis nerve (14), the tibialis anterior muscle (15), the extensor hallucis longus muscle (EHL) (16), the peroneal nerve (17), the gastrocnemius muscle (18) and the posterior tibial nerve (19), in accordance with one the preferred embodiment.

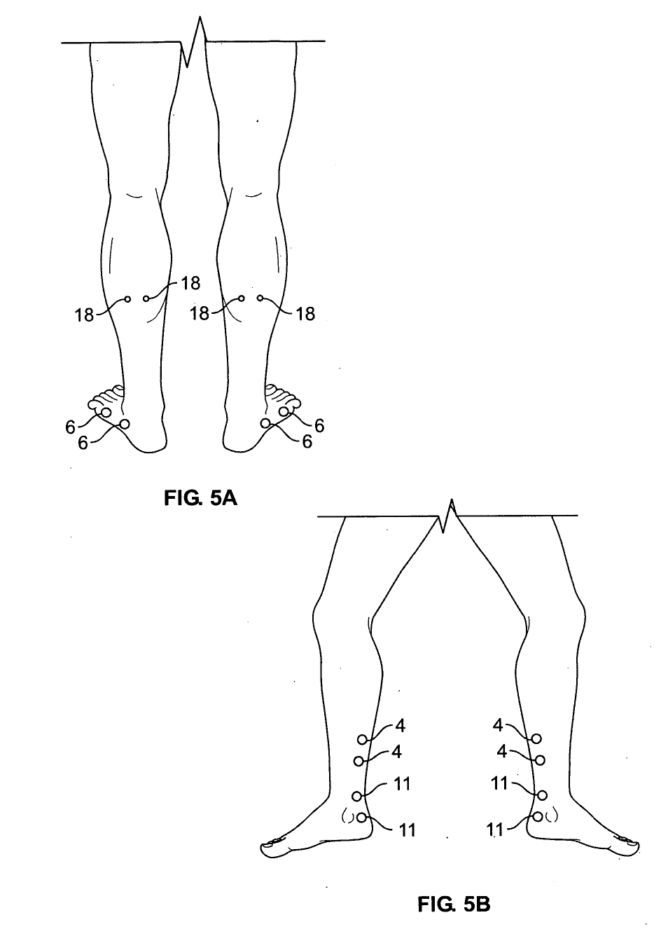

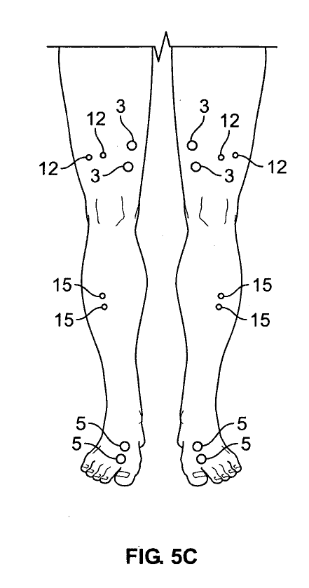

In yet another preferred embodiment of the inventive stocking, shown in FIGS. 5A, 5B and 5C, the stocking has a combination of small and large apertures suited to a montage in which a combination of both snap-on and needle electrodes is utilized. FIGS. 5A, 5B and 5C show, respectively, a posterior view, an inner lower limb view and an anterior view of a stocking having apertures for placement of both snap-on and needle electrodes over the gastrocnemius muscle (18), at spinal roots S1 (6), L4 (4), L3 (3), L5 (5), the posterior tibial nerve (11), the quadriceps muscle (12) and the tibialis anterior muscle (15), in accordance with one of the preferred embodiments of the invention.

The invention contemplates the use of the inventive stocking in conjunction with all types of electrode technology, and it will be understood by those skilled in the art that the aperture size may be of any size suited to and/or appropriate for any type of electrode. Other designs of apertures for guidance and placement of electrodes may be desirable. It will be understood by those skilled in the art therefore that the inventive stocking is not to be limited to any particular design, nor to any particular shape, style or size of aperture, as the instant invention contemplates the use of all types of electrodes for receiving or transmitting data, or for stimulating the skin of a subject.

It will be also understood by those skilled in the art that the apertures of the stocking may be designed for differentiating the apertures, such as color-coding and the like, the apertures marked so as to designate placement of a particular electrode at a particular site on the subject. Alternatively, the stocking apertures may be unmarked or undesignated, so that all the apertures look alike except for the size of the apertures, or alternatively, all the sizes of the apertures may be alike. In one embodiment of the inventive stocking apertures designed without placement marking be suitable marked by the user via coloring the stocking with a marker pen, or by attaching appropriate guidance markers thereon.

Table 1 illustrates a color correlation schedule, based on embodiments of the invention shown in FIGS. 2-5, in which the color of an aperture designates an electrode placement at a position on the subject's body. Color coding may be affixed to the stocking by a manufacturer or the stocking may by any means such as hatched colored stitching around the aperture, or it may be by another means for marking on, around or adjacent to the aperture a color designating the electrode. A color chart providing information such as is shown in Table 1, may be provided with the color-coded stocking. In such a chart, free electrodes may be provided for, leaving the color optional, for example for motor nerve conduction. Alternatively, the chart may be provided that is capable of receiving removable or erasable color coding for use with multiple different styles of stocking. It will be obvious to one skilled in the art that any type of designation of apertures in the stocking or undesignated apertures may be used with any kind of pre-designed chart or a re-usable chart such as just described.

TABLE 1

| | Root, nerve or muscle | Color-coding |
|---|---|---|
| 2 | L2 Nerve Root | red |
| 3 | L3 Nerve Root | blue |
| 4 | L4 Nerve Root | yellow |
| 5 | L5 Nerve Root | orange |
| 6 | S1 Nerve Root | black |

| | |
|---|---|
| 7 | free electrodes: color optional |
| 8 | for motor nerve |
| 9 | conduction |

| | | |
|---|---|---|
| 10 | Peroneal Nerve | black |
| 11 | Posteria Tibial Nerve | violet |
| 12 | Quadriceps Muscle | red |
| 13 | Lateral Femoral Cutaneous Nerve | orange |
| 14 | Vastus Lateralis Muscle | blue |
| 15 | Tibialis Anterior Muscle | yellow |
| 16 | Extensor Hallucis Longus (EHL) Muscle | black |
| 17 | Peroneal Nerve | green |
| 18 | Gastrocnemius Muscle | brown |
| 19 | Posterior Tibial Nerve | violet |

The instant invention also contemplates the use of the inventive stocking with one or more wireless electrodes, in which the stocking apertures are used as described heretofor, but now for placement of wireless electrodes or biosensor electrodes.

In a highly preferred embodiment, the inventive stocking or sleeve is a component of a neurophysiological procedure wherein procedures comprising one or more of stimulation of a subject, recording bio-potentials from a subject, and monitoring changes in a subject, is carried out in real-time.

As an exemplary procedure, evoked potentials are monitored in real-time, by recording far-field potentials generated distant from a stimulation site. For example, the posterial tibial nerve is stimulated in the lower limb, and its volley or electroconductive changes can be measured at a recording electrode, which could placed over any far-field volume conductor such as the posterior spinal column, the cerebral cortex, or the lumbar sacral spine. The window in which recording over the biosensor is being made, is time-locked to the delivery of the stimulus. From the point of stimulation of the lower extremities to recording a far-field bio-potentials over the posterior cervical spine or cerebral cortex, the recording window of a lower extremity nerve, for example, the posterial tibial nerve, or of a dermatome, is 100 milliseconds (msec). The recording window from the point of stimulation at the upper extremities (the median nerve) to the cervical spine or cerebral cortex, is 50 msec. In compound action muscle potentials in which recording is being made from the muscle, the time-window is 20 msec. In neurogenic evoked potentials (in which recording takes place at a nerve, and stimulation may be of any segment proximal to where a signal is being recording from), the time window is 30 msec. For ECG, and EMG, the time window is 100 msec.

For example, if at time t=zero, a stimulus is delivered, a recording is captured over the lumbar sacral spine in about 40 seconds. Typical recording time windows are shown in Table 2.

TABLE 2

| | | Recording |
|---|---|---|
| Stimulating | Recording | Window (msec) |
| Posterial tibial | | 100 |
| median | | 50 |
| | muscle | 20 |
| | NEP | 30 |
| | ECG, EMG | 100 |

In one embodiment of the invention, a support stocking is manufactured with apertures that are color-coded according to placement of electrodes for an all snap-on electrode montage. The stocking is put on the leg of a subject undergoing a procedure for measuring function of the peroneal and posterior tibial nerves, and at spinal roots L4, L2, L3 and L5. Once the stocking is fitted on the leg of the subject in the normal way a stocking or medical hose is fitted onto the leg, the apertures become positioned such that it is easy for the practitioner to see the positions for placement of the electrodes. The aperture for the peroneal nerve is colored black and indicates the position for placing a stimulating electrode, also colored black, to stimulate this nerve. In similar fashion, the aperture indicating the site for stimulating the posterior tibial nerve is colored violet, correlating with a violet colored stimulating electrode. In similar fashion the apertures indicating the position for stimulating spinal roots L4, L2, L3 and L5, are respectively colored yellow (L4), red (L2), blue (L3) and orange (L5), each correlating with stimulating electrodes similarly colored.

In a second example of the inventive approach, the inventive stocking is worn by a subject during a procedure using an all needle electrode montage, in which the quadriceps muscle, the lateral femoral cutaneous nerve, the vastus lateralis muscle, the tibialis anterior muscle, the extensor hallucis longus muscle, the peroneal nerve, the gastrocnemius muscle and the posterior tibial nerve are percutaneously stimulated or recorded. When fitted on the leg of the subject, the apertures in the stocking are positioned for receiving and placing needle electrodes at the quadriceps muscle, the lateral femoral cutaneous nerve, the vastus lateralis nerve, the tibialis anterior muscle, the extensor hallucis longus muscle, the peroneal nerve, the gastrocnemius muscle and the posterior tibial nerve. The apertures are colored, respectively (as shown in Table 1), red for placement of an electrode recording the quadriceps muscle, orange for stimulating the lateral femoral cutaneous nerve, blue for the vastus lateralis nerve, yellow for the tibialis anterior muscle, black for the extensor hallucis longus muscle, green for the peroneal nerve, brown for the gastrocnemius muscle and the violet for the posterior tibial nerve. The correlating electrodes are similarly colored accordingly. In this manner, the practitioner can rapidly and correctly attach the appropriate electrode.

What is claimed is:

1. A manufactured stocking or sleeve for wearing or wrapping around a part of a subject's body during a neurological procedure for guiding placement of electrodes, having positioned apertures corresponding to a specific electrode montage, wherein the electrodes are placed on the surface or just beneath the surface of the skin of a subject at the positions of the apertures.

2. The stocking or sleeve of claim 1, wherein the stocking is worn on or is wrapped around a part of a subject's body selected from the group consisting of a leg, an arm, a hand, an upper part of the trunk and a lower part of the trunk.

3. The stocking or sleeve of claim 2, wherein the apertures are marked or distinguished to aid placement of electrodes, or wherein the apertures are markable.

4. The stocking or sleeve of claim 3, wherein the apertures are color-coded.

5. The stocking or sleeve of claim 1, wherein the stocking or sleeve is a component in a system for monitoring and evaluating a neurophysiological response in a mammalian subject, comprising means for collecting, analyzing, correlating and reporting electroneurophysiological data in real-time.

6. A system for comparing and evaluating in real-time bio-potentials elicited by a stimulating electrode at a stimulation site on a mammalian subject, the system comprising:
a) hardware means for eliciting a signal from a first stimulation site on a subject, receiving and amplifying the signal, and recording a waveform signal;
b) hardware means for automatically digitally converting the waveform signal and software means for assigning numeric values for the absolute amplitude and absolute latency of the waveform signal;
c) hardware and software means for replicating the steps a) and b) to obtain a series of replicated digitally assigned waveform data for the first stimulation site;
d) software means for mathematically conditioning the series of replicated digitally assigned waveform data, obtaining a validated mean value for the waveform data for the first stimulation site, then comparing the validated mean value with protocol-specific and subject-specific normal waveform data, assessing the comparison and noting the deviations of the waveform data from normal data;
e) software means for performing steps a) to d) and serially comparing and evaluating in real-time the changes in the waveform data and saving the comparisons and changes as a function of time.

7. The system of claim 6, further comprising hardware and software means for carrying out steps a)-e) with respect to two or more different stimulations sites on the subject, for comparing and evaluating the changes in the waveform data and saving the serially obtained comparisons and evaluations as a function of time.

8. The system of claim 6, further comprising hardware means for recording the stimulation signal at a subcortical recording site on the subject.

9. The system of claim 6, wherein a) further comprises a sleeve or stocking for guiding placement of electrodes, having positioned apertures corresponding to a specific electrode montage, wherein the electrodes are placed on the surface or just beneath the surface of the skin of a subject at the positions of the apertures.

10. The system of claim 9, wherein the elicited bio-potentials are selected from the group consisting of electrocardiogram data, electromyogram data and evoked potentials.

11. The system of claim 9, wherein the evoked potentials are somatosensory evoked potentials or dermatomal somatosensory evoked potentials or both.

12. The system of claim 9, further comprising software means for correlating more than one set of bio-potentials from the same subject selected from the group consisting of electrocardiogram data, electromyogram data, somatosensory evoked potentials and dermatomal somatosensory evoked potentials.

13. A bio-potential signal acquisition system comprising the hardware and software means of claim 9.

14. A method of comparing and evaluating in real-time bio-potentials elicited by a stimulating electrode at a stimulation site on a mammalian subject the method comprising:
a) eliciting a signal from a first stimulation site on a subject, receiving and amplifying the signal, and recording a waveform signal;
b) automatically digitally converting the waveform signal and assigning numeric values for the absolute amplitude and absolute latency of the waveform signal;
c) replicating the steps a) and b) to obtain a series of replicated digitally assigned waveform data for the first stimulation site;
d) mathematically conditioning the replicated digitally assigned waveform data, obtaining a validated mean value for the waveform data for the first stimulation site, then comparing the validated mean value with protocol-specific and subject-specific normal waveform data, assessing the comparison and noting the deviations of the waveform data from normal data; and
e) performing a series of further trials in the manner of steps a) to d) and serially comparing and evaluating in real-time the changes in the waveform data and saving the comparisons and changes as a function of time.

15. The method of claim 14, further comprising serially performing the steps a) to e) with respect to two or more different stimulation sites on the subject, comparing and evaluating the changes in the waveform data and saving the serially obtained comparisons and evaluations as a function of time.

16. The method of claim 14, wherein in step a) the waveform signal is recorded at a subcortical recording site on the subject.

17. The method of claim 16, further comprising placing electrodes on the subject by means of a stocking or sleeve having positioned apertures corresponding to a specific electrode montage, wherein the electrodes are placed on the surface or just beneath the surface of the skin of a subject at the positions of the apertures.

18. A chart for use with the stocking or sleeve of claim 1, or with the system of claim 9, or in the method of claim 17.
